(19) United States
(12) Patent Application Publication
(10) Pub. No.: US 2014/0005468 A1
(43) Pub. Date: Jan. 2, 2014

(54) Method of Training A Living Body To Not React To Substances

(71) Applicant: Dawn Nowlin, Leesburg, VA (US)

(72) Inventor: Dawn Nowlin, Leesburg, VA (US)

(21) Appl. No.: 14/004,257

(22) PCT Filed: Mar. 12, 2012
(86) PCT No.: PCT/US2012/028832

§ 371 Date: Sep. 10, 2013

(30) Foreign Application Priority Data
Mar. 10, 2011 (US) 13044986

(51) Int. Cl. A61M 21/00

(52) U.S. Cl. CPC A61M 21/00
USPC 600/28

(57) ABSTRACT

A method of conditioning a living body of a patient to associate the positive effects of sensory stimulation of the sympathetic ganglia with digital audio representations of offending substances to modify errant interpretations of various systems involved in a reaction to ultimately cease or reduce negative reactions to such substances. In addition, a patient's sensitivities are treated by using digital representations, preferably provided via a computer, to represent the actual substances in order to engage the multimodal functioning of the brain. Sensory stimulation is used in conjunction with the digital audio signals to condition the body to react more appropriately to the substance. Motor activity is measured before treatment to show an action schema of “retreat”. Stimulation is administered to the sympathetic ganglia until the motor activity is restored to normal capacity, indicating the perception of the substance has been modified.

### FIELD OF THE INVENTION

The present invention is a method of conditioning a living body of a patient to associate the positive effects of sensory stimulation of the sympathetic ganglia as the primary stimulus with digital audio representations of offending substances as the secondary stimulus to modify errant interpretations of bodily systems involved in a reaction to ultimately reduce negative reactions to such substances. In addition, the method of the present invention serves to improve a patient's symptoms caused by a psychosomatic component, which contributes to sensitivities, by taking advantage of multimodal functioning of the brain, thus using audio digital representations of the substance being treated.

### BACKGROUND OF THE PRESENT INVENTION

Sensitivities are the result of bodily systems reacting inappropriately to harmless, often naturally occurring substances, which can affect virtually every part of the body. These reactions are abnormal.

A large percentage of reactions to harmless substances do not involve the immune system. Such reactions that do not involve the immune system are referred to as sensitivities and often include a psychosomatic component as a contributing factor. The symptoms of sensitivities may be as pronounced and can even be as severe as those of true allergies, with no immune involvement. It has only been recently recognized that sensitivity-related illness (SRI) may involve various organ systems and evoke wide-ranging physical or neuropsychological manifestations.

The inventor has concluded that the body's response to a substance may depend on the interpretation or perception of that substance. If the body perceives the substance as harmful, it will react in an inappropriate manner, with sympathetic hyperactivity in the case of organ system involvement. If the substance is benign, an inappropriate reaction is the result of an errant perception of the substance leading to sensitivity reactions.

This invention is not based on treating the immune system, but instead approaches the physiological reaction of sensitivities by addressing the perceptual error stemming from a psychosomatic relation to the substance. The invention takes advantage of adaptive mechanisms of the body utilizing a conditioning method that enables the human brain and cognitive processes to adapt to the internal and external milieu. Traditional cognitive studies typically depend on stimulus only; however, this invention utilizes stimulus and context perceived by the subject.

The mechanism of the invention is supported by studies on mirror neurons and associated motor activity. In 1996, Giacomo Rizolatti of the University of Parma in Italy discovered a subset of neurons from the brain's premotor cortex found in macaque monkeys which fired when the monkey performed a certain action.¹ Those neurons also fired when the monkeys simply observed the same action being performed by other monkeys. In 2006, a team led by Lisa Aziz-Zadeh from the University of Southern California, discovered, by using fMRI, that not only were cells activated by observing an action, they also responded when the subject read about the action.² Aziz-Zadeh also found that the motor neurons were even activated by the sound of an action, concluding that mirror neurons are multimodal. The studies have shown that perception, language and action are closely intertwined. These studies on mirror neurons have also shown that words, phrases or even sounds representing action or meaning do have an effect on motor processes and neural activity. The invention takes advantage of this phenomenon and utilizes both auditory and visual representations of the offending agent in treatment.

In addition to working with the brain's multimodal capabilities of perception and interpretation, the invention also takes advantage of motor activity that corresponds to meaning, specifically the interpretation of an offending agent. Studies conducted by Arthur M. Glenberg et al³ have used brain imaging techniques to show that during processing of language, material with content related to different effectors, effector-specific sectors of the premotor and motor areas become active. Behavioral and neurological studies have also shown a modulation of motor responses related to content of language material. Glenberg and Kaschak⁴ have shown that sentences are understood by creating simulation of the actions that underlie them and motor neurons, creating an “action schema”. In addition, a theory has been proposed by Lindeman and Abramson⁵ combining simulation with Lakoff's analysis with conceptual metaphors. The purpose of their research is to look at causal mechanism that links cognitive and somatic elements of depression. They found that, in fact, the inability to alter events (leading to depression) is conceptualized metaphorically as motor incapacity and that this leads to peripheral physiological changes consistent with motor incapacity. Thus, the action schema for depression is “motor incapacity”.

Another example for embodiment of metaphorical conceptions was demonstrated by Glenberg et al. Such concepts as delegating responsibilities or communicating information, resemble, for example, statements like “she gave him the idea”. These concepts involve mental simulation of transferring physical objects by hand from one person to another. In addition, they also found that weak motor impulses to the hands also occurred during the simulation.⁶ Lakeoff and Johnson⁷ found that cognitive processes responsible for conjuring the metaphorical concepts and linking abstract ideas with sensorimotor experience need not be conscious or voluntary.

In a conceptual framework, the brain may categorize a perceived irritant with other dangers such as “poison” or “fire”. The action schema for motor activity is not motor incapacity, as in the case of depression, but instead motor activity representing “retreat” or “avoid”. Therefore, there is a measurable change in motor activity that represents the action schema of “retreat”. This invention measures for action schema simulating a motor activity resembling the process of retreating.

Motor performance is measured from reading or hearing a word that represents an offending agent. The instrument detects a weakening of motor activity by using a comparison of the upward force of the subject's index finger against a fixed and force-applied object and then downward movement to test motor integrity. A degradation of motor resistance when being presented with a representation of an offending agent would indicate an action schema of “retreat” or “avoid”.

By presenting a language-based representation of the offending substance to the patient while simultaneously introducing a sensory stimulus, the body associates the positive stimulus with the benign substance and alters its perception, interpreting the substance as beneficial. The inventor has found that the substance being treated may be introduced to the subject as an audio digital representation of the substance provided by a computer or other device. The multimodal processes of the brain interpret the representation in a similar fashion as coming into contact with the actual substance. The inventor has found that cognitive processes can interpret the representation of the substance used in treatment for the conditioned effect.

The first known study on the phenomenon of conditioning was in 1902, by Ivan Pavlov, demonstrating that conditioned reflexes may be learned either by repetitive stimuli or by associating two stimuli. The conditioning process causes a learned behavior that responds to the associated stimulus based on the newly interpreted meaning of that stimulus. Pavlov concluded that a connection was made in the nervous system linking an environmental stimulus to an unconditioned reflex, transforming the reflex into a conditioned reflex, activated by an external stimulus. This invention utilizes sensory stimulus to the sympathetic ganglia as the primary stimulus and contextual audio digital signals of an offending agent as the secondary stimulus to create a new association or interpretation of the treated substance.

The process which Pavlov described as a formation of conditioned reflexes is essentially a translation of messages from a psychological sign system into messages of a system of somatic signs and vice versa. With this translation, a connection develops between the psychological and the somatic levels. The stimulus to the body and use of contextual representations for the mind or cognitive processes can be integrated and, in fact, are connected.

Pavlov concluded that in animals there exists only a first system of signals of reality, allowing the brain to receive and analyze stimuli within the organism as well as outside the organism. In humans, there exists both this first level as well as a second level of signals: language or symbols. Words and symbols can function as stimuli in humans, so real and effective, that they can mobilize humans just as a concrete stimulus. Words are symbols, abstractions; the conditioned stimulus can be generalizable.

The digital signal used in treatment is initially created from a textual representation. This textual representation is converted to an audio digital signal and transmitted repeatedly during treatment. The invention engages both conscious and unconscious processes by transmitting the digital signals at low decibels that are still audible. The digital audio signal is transmitted via speaker embedded in a cuff or mat that the patient lies on. (Sources:

1. Rizzolatti, G., Fadiga, L., Gallese, V., & Fogassi, L. (1996) Premotor cortex and the recognition of motor actions. Cognitive Brain Research, 3 131-141

2. Aziz-Zadeh, L., Wilson, S. M., Rizzolatti, G., & Iacoboni, M. (2006) Embodied semantics and the premotor cortex: Congruent representations for visually presented actions and linguistic phrases describing actions. Current Biology, 16, 1818-1823

3. Glenberg, A. M., Sato, M., Cattaneo, L., Riggio, L., Palumbo, D., & Buccino, G., (2008) Processing abstract language modulates motor system activity. The Quarterly Journal of Experimental Psychology, 61 (6), 905-919

4. Glenberg, A., M., & Kaschak, M. P. (2002). Grounding language in action. Psychonomic Bulletin & Review, 9(3), 558-565

5. Lindeman L., Abramson L., The Role of simulation of motor incapacity in depression. J. Cogn Psychotherapy 2008, 22:228-249

6. Lindeman L., Abramson L., The Role of simulation of motor incapacity in depression. J. Cogn Psychotherapy 2008, 22:228-249

7. Lakoff, G., & Johson, M. (1999) Philosophy in the flesh: The embodied mind and its challenge to Western thought. New York: Basic Books)

### SUMMARY OF THE PRESENT INVENTION

The method of the present invention is as follows: Motor activity is measured from the subject's index finger prior to transmitting audio digital signals. Baseline motor capacity is established. A digital audio representation (optional visual textual representation may additionally be used) of the offending agent is presented to the subject. Motor activity is re measured for the action schema of “retreat”, which results in a degradation of motor capacity. Subject is then treated by presenting the digital audio representation which represents the substance the body is inappropriately reacting to, preferably via a computer playing a digitized sound, stimulation from motor vibration is administered at the location of the sympathetic ganglia of the organ systems involved in a reaction. Stimulation of these areas has been shown to temporarily improve the afferent and efferent function of the effected organ systems and to alleviate sympathetic hyperactivity. The simultaneous stimulation of sympathetic ganglia with the cognitive perception of the signal teaches or reconditions the body to perceive the substance as beneficial as it becomes associated with the positive stimuli and thereby modifies the physiological reaction. Stimulation is administered to the sympathetic ganglia until the motor activity is restored to normal capacity, indicating the perception of the substance has been modified. The present invention is designed to engage the multimodal functioning of the brain primarily through audio signals but also through visual textual signals.

The mechanism underlying the effect of the treatment is a conditioned association that creates a “coupling of meaning” whereby the substance is “coupled” with the positive effect of the stimulus. The modified perception of the harmless substance alters the behavior of the effected system(s). The therapeutic stimulation used in conjunction with the transmission of digital signals conditions the body to alter its perception of a harmless substance. The transmission of digital signals has no effect or therapeutic value to the body. It is only the stimulation in conjunction with the presentation of the digital representation that allows for a resolve in the physiological error.

The inventor has discovered that the human body can inappropriately react to a vast number of substances including stimuli, such as heat or sunlight. Therefore, a database, preferably maintained by a computer, is used to include a large number of substances and further breaks the substances down into components. This methodology allows for superior precision in addressing the exact substance in which the body is reacting.

A joint study was conducted in Seoul, Korea in 2010, authorized by the inventor of the present invention and under the direction of Seung-Ho Ph. D. of Kyung, Hee University in Seoul, Korea. The study was conducted to test the efficacy of utilizing digital representational signals in the conditioning process for the treatment of sensitivities. A total of 85 cases were treated for a range of sensitivities to shrimp, crab, cucumber, peach, chrysalis, metals, garlic, pineapple, stone fruit, red wine, peppers, coffee, dust/dust mites and cat dander. Patients received between 1-3 treatments during the study. At least 24 hours of interval was required between treatments for each subject. Subjects ranged in age from 9 to 65 years with 49 females and 36 males, all of Korean or Japanese decent. Inclusion criteria were that subjects must have recently experienced sensitivity reactions. The reactions were to be clearly observed within a 24 hour period. Reactions were not to he present with avoidance, ruling out additional contributing factors. Exclusion criteria included any symptoms that required long term treatment due to the time limit of the study. No control group was employed. No blind test was executed. However, a blindness in the assessment of outcomes was applied.

To assess the efficacy of the treatment, patients reported improvement On a scale from 0-100% with 0=no improvement and 100%=complete resolution. If the patient reported 100% resolution, no symptoms occurred upon exposure after treatment. Reports were collected as subjects visited the test hospital and challenge tests were completed by subjects following 24 hours after treatment. Neither blood tests nor skin tests were included as cases were primarily related to food substances with psychosomatic involvement as opposed to true allergies with immune involvement.

Patient reporting and visible evidence is considered to be more clinically relevant, therefore the most accurate test for treatment results was to challenge the offending substance after treatment. For the purposes of this study, symptomatic reactions from the effective organ systems were the focus for treatment results.

The results of the study showed that 61% of the cases responded with substantial improvement with some cases showing complete resolution of symptoms. Subjects could experience continued, exposure without the previous symptoms. Moderate improvement was reported by 22% of the cases and little or no improvement was reported by 16% of the cases. Considering a generally accepted value of placebo effect of 20% in medical treatments, the study confirmed that the treatment produced a clinically significant result. Three months after treatment 46% of cases who responded to the follow up reported a sustained result, 38% reported either an increase in symptoms or return to the original symptoms and 15% reported an improvement from the, initial results. This invention is not intended for use on cases of anaphylaxis or life threatening sensitivities.

Additionally, a mat capable of providing parasympathetic stimulation and audible transmission of sound may be employed by the method of the present invention, in lieu of a cuff. The patient would preferably lay his or her back on the mat. The mat contains a speaker, which is preferably embedded or integrated into the mat, which contains vibratory motors designed for stimulation.

### DETAILED DESCRIPTION OF THE EMBODIMENT(S) OF THE PRESENT INVENTION

The present invention is a method that serves to treat digital audio representations associated with a specific substance in order to ultimately condition the body's natural physiology to accept such specific irritant that initially caused the sensitivity reaction. In one embodiment, sensory stimulation is administered to locations near the sympathetic ganglia of the organ systems, along each side of the spine, with the use of vibratory motor stimulation. This stimulation is utilized to temporarily improve the afferent and efferent function and alleviate sympathetic hyperactivity in the major organ systems which are overreacting. Meanwhile, the digital representation specific to the substance found to cause the irritation is played (and optionally presented in text on the computer monitor). The combination of the cognitive perception of the digital audio signals and the stimulation of the sympathetic ganglia teaches or conditions the body to not react to that particular substance. In other words, the different digital signals, provided via a computer, that represent an irritant transmitted at the same time as applying a positive stimulus ultimately trains the body to associate the irritant with positive benefit.

The present invention can proceed as follows. First, a speaker is positioned in the proximity of the patient (via cuff or embedded into the stimulation mat). It should be noted that the patient also might be referred to as person or subject. In one embodiment, the speaker may be placed on the underside of an arm cuff. The arm cuff would then be wrapped around the arm of the patient or positioned in the vicinity of the patient's ear. The cuff interfaces with a computer via a USB port. Motor activity is measured from the subject's index finger prior to transmitting audio digital signals. Baseline motor capacity is established. Then, a digital audio signal is transmitted, preferably via a computer. The signal being transmitted is representative of what the actual potential offending agent is purported to be. This means that the sound literally can be words identifying the purported irritant.

While the signal is transmitted toward the patient, a sensory stimulation is applied to the sympathetic ganglia of the major organ systems utilizing vibratory motors that run alongside the vertebrae. Additional stimulation may be applied to specific sympathetic ganglia corresponding to organ systems which present the prevalent symptoms until the motor activity is restored to normal capacity, indicating the perception of the substance has been modified. The stimulation, which causes an alleviation of hyperactivity, along with the representation of the substance has the effect of conditioning the patient's body to perceive the irritant as a physical positive, which in turn will diminish the inappropriate reaction.

The mat that is utilized in this present invention is a thin foam mat that encases a series of vibratory motors. The series of vibratory motors are, in one embodiment, arranged in two columns which are each 4 feet (121.92 cm) in length; there is a 3 inch (7.62 cm) separation between the two columns. The series of vibratory motors provides a gentle percussion when activated by the practitioner, via the software program. The mat is placed on a treatment table and the patient lies on the mat so that the vibratory motors make contact at the location of the paraspinal muscles, specifically between the spine and the paraspinal muscles of the patient. When activated, the device stimulates the sympathetic ganglia areas with a gentle percussion. The percussion occurs while the sound is transmitted toward the body of the patient, as described above. And thus, it would be preferred that in the mat is the speaker, located at the top of the mat (near where the patient's head rests) that transmits the digital audio signals, so that the low audible sound may still be heard by the patient to engage the conscious mind in the conditioning. The mat replaces both the need for the positioning of the speaker in the arm cuff noted above, and the stimulation administered to the sympathetic ganglia by the doctor/practitioner would not be necessary because of the percussion from the mat. It should be understood that the mat and the cuff are of conventional design. The present invention is designed to engage the multimodal functioning of the brain primarily through audio signals but also through visual textual signals shown on the monitor of the computer from the software program.

According to the present invention, it should be understood that the doctor conducts a thorough patient intake to assess what substance or substances are causing the inappropriate reaction. Once the substance for treatment is determined, treatment is administered. Motor activity is measured from the subject's index finger prior to transmitting audio digital signals. Baseline motor capacity is established. The signal representing the substance is played to the patient via the software program. At the same time, the motors of the mat are activated in order to stimulate the sympathetic ganglia to alleviate the hyperactivity of the organ systems. It should be understood that the mat of the present invention performs the stimulation, and not the practitioner. An overview of the mat apparatus and overarching system of the present invention and how it is used is outlined below

The below describes an apparatus designed to test the state of a subject when audio or visual textual signals are presented. The instrument will detect a weakening due to a “retreat” action schema caused by textual signals representing a substance that is perceived as a threat to the system. The loss of integrity will be detected through the measurement and comparison of the upward force of the patient's index finger against a fixed and force-applied object. A degradation of the force when digital audio signals are played will confirm an action schema of “retreat”. The apparatus will measure upward force, set baselines, apply calculated downward pressure, and determine positive and negative conclusions and/or trends. It is to be understood that motor activity is measured before treatment to show an action schema of “retreat”. Stimulation is administered to the sympathetic ganglia until the motor activity is restored to normal capacity, indicating the perception of the substance has been modified. It should be noted that stimulation may be administered manually, or via the mat.

The components of the work product include an electro-mechanical apparatus, a computer control/assessment application, and informational signal delivery methods—computer screen, speakers, and mat for stimulation of sympathetic ganglia.

### Diagnostic Device for Motor Activity for the Method of the Present Invention

This section will outline the basic patient diagnostic procedure that the system of the present invention is designed to support.
1. A patient positions their hand palm down on or in the apparatus
2. Apparatus downward force mechanism is designed to contact the patient at the first knuckle (from tip of finger) of the patient's index finger with an inflexible, surface. Apparatus allows some adjustment in actual contact point on patient's finger.
3. The patient raises their finger lifting the slightly lowered downward force mechanism up to the physical stop/limitation point
4. When the patient receives an indication (LED) that the signal is transmitted, they further press upward to their limit. This signal may originate from a manual pressing of an interface control by the administering tester/practitioner.
5. The practitioner will be able to select how the agent signal will be transmitted to the patient. This can be an audible signal played on the computer or through additional speakers attached to the computer and embedded into a cuff or the mat. The audible signal can be played at normal speed or can be played at an increased speed. The practitioner can select the signal to be displayed on the computer screen for the patient to read for visual imposition of the agent. This visual representation of the agent can be done uniquely or coincidentally with audible transmission via the computer or the mat. Finally, the signal can be transmitted audibly to the patient via speakers embedded in the mat. (Defined below)
6. A pressure measurement device built into the lowering mechanism of the apparatus will indicate when the patient reaches a sufficient level of force/pressure.
7. When sufficient force/pressure is met, another color LED will indicate this and at the same time initiate the downward force mechanism to increase downward force.
8. The patient will attempt to hold the lock. Lock is defined by two opposing forces meeting in an equalizing or near equalizing position.
9. When a maintained lock is achieved:
   a. System will indicate that lock has been maintained (via a lit LED).
10. If a lock condition is not maintained, system will detect this condition through one or both of the following:
   a. Lowering of the finger by downward force mechanism
   b. Reduction of upward force applied by finger of the patient.
11. System will indicate perturbation (could be an LED).
1. The apparatus outlined that is employed with the method of the present invention is preferably a single unit and will consist of the following components:
   1.1. Base—patient's hand rests on this to provide accurate measurement of opposing exerted forces.
   1.2. Downward force mechanism
      1.2.1. Designed to allow upward pressure to be exerted against it by any of the patient's fingers (not including the thumb). Typically this will be the patient's index finger.
      1.2.2. Designed to provide a contact surface for the patient's finger at the first knuckle of the finger (from tip of finger). Actual contact point on patient's finger will be able to be varied.
      1.2.3. Designed to achieve rigid contact with the patient's knuckle with a hard, inflexible, non-deforming surface.
      1.2.4. Accommodates various hand and finger sizes.
      1.2.5. Allows upward movement of at least 1 inch (2.5 cm) between finger contact and “lock” position.
      1.2.6. Lock position is enforced by a physical stop. “Physical stop” is defined as a mechanical limit to movement and might be implemented through a variety of physical or electromechanical means.
   1.3. Force gage
      1.3.1. A piezoresistive film pressure transducer is used to measure force
      1.3.2. Force gage provides a maximum activation pressure sensitivity of approximately 50 grams
      1.3.3. Force gage provides a minimum activation pressure sensitivity of approximately 10 grams (requires at least 10 grams to initiate force readings).
      1.3.4. Force gage is preferably able to measure forces up to 2 kg (4.4 pounds)
      1.3.5. The relative/approximate pressure value sensed by the apparatus control software may be viewed on a computer screen readout.
   1.4. Servo motor—
      1.4.1. A small, incremental downward movement/pressure to downward force mechanism.
      1.4.2. Can exert approximately 2 kg (4.4 pounds) downward pressure.

### Apparatus Electrical Definition

1. Apparatus obtains its power from the computer USB port. No external power source is required. This requires custom driver software. Power consumption may exceed the power (current) provided in certain devices for example USD hubs and some sub-notebooks, IPad or mobile devices which happen to have a USB port. The guiding model of ‘current available’ is a desktop computer where the apparatus is directly connected to a motherboard connector in USB format. A later model laptop with fully charged battery and power supply should also be sufficient.
2. Control board—
   2.1. Provides power and control signals to servo motor
   2.2. Provides servo motor control interface to computer
   2.3. Powers force gage
   2.4. Receives force gage output
   2.5. Converts force gage analog output signal to digital
   2.6. Transmits force gage readings to computer
3. LEDs—unit will have the following LEDs or multi-color LED that will alert patient and practitioner of test status
   3.1. Begin Signal Transmission (patient will lift plate up to physical stop)
   3.2. Ready position met
   3.3. Signal transmission has begun and downward force will begin
   3.4. Test completed (patient can relax)

### Apparatus Definition—COMPUTER Application

1. Include a USB driver for the Computer recognition of, and ability to communicate with, the apparatus of the present invention.
2. The Computer can communicate with only a single apparatus.
3. No user management system is required. Application does require login or password protection.
4. A database is required for data storage.
5. Session/test management
   5.1. Create a test session
      5.1.1. Test session name (text input)
      5.1.2. Test type (text input)
      5.1.3. Notes/Comments (text input)
   5.2. Start a test session
      5.2.1. Begins/opens the above-created test session
      5.2.2. The practitioner selects an agent signals from the agent library. This agent may be presented to the patient in one of several ways.
      5.2.3. The practitioner selects agent distribution method
         5.2.3.1. Audible via Computer
         5.2.3.2. Visual via Computer
         5.2.3.3. Audible via Computer and Visual via Computer
         5.2.3.4. Audible via Mat
         5.2.3.5. Audible via Mat and Visual via Computer
      5.2.4. Practitioner sets vibration parameters for Optional Mat
      5.2.5. Practitioner initializes the session
      5.2.6. PC application sends “begin session” command to apparatus
   5.3. Run test session
      5.3.1. Detects Initial Continuous force against force gage (indicates finger is raised) for 2 seconds <Initial Continuous Force Value>
      5.3.2. Calculates and stores average Initial Continuous force value
      5.3.3. Provides practitioner with “button” to indicate transmission has begun
      5.3.4. Lights Begin Signal Transmission LED
      5.3.5. Reads force gage for achievement of Threshold Met pressure threshold
      5.3.6. When Threshold Met pressure threshold is met for 0.5 seconds, (subject presses upward into the barrier until a certain level of force is attained) this lights the Threshold Met LED.
         5.3.6.1. Threshold Met LED indicates Threshold pressure is met and that downward pressure will begin. Threshold Met LED stays lit for 2 seconds
      5.3.7. After lighting Threshold Met LED, delays for 1 second and then activates a servo motor to begin Lock Maintained downward force
      5.3.8. Reads force gage
      5.3.9. (If Lock Maintained downward force value is achieved), lights Test Completed LED for 5 seconds and stops servo motor downward force
      5.3.10. If Lock Maintained force value) is not achieved after 2 seconds, lights Test Completed LED for 5 seconds and stops servo motor downward force coincidental with lighting of LED.
   5.4. End a test session
      5.4.1. Lock Maintained or Lock Lost status ends the current test session
      5.4.2. Stores all data in session file
      5.4.3. Displays current session results on screen
         5.4.3.1. Initial Continuous force value
            5.4.3.1.1. Setting
            5.4.3.1.2. Measured
         5.4.3.2. Threshold Met force value
            5.4.3.2.1. Setting
            5.4.3.2.2. Measured
         5.4.3.3. Lock Maintained force value
            5.4.3.3.1. Setting
            5.4.3.3.2. Maximum measured
         5.4.3.4. Lock Lost force value
            5.4.3.4.1. Setting
            5.4.3.4.2. Maximum measured
      5.4.4. The ability to view in graph form the force measurements acquired during the lock test process.
   5.5. Session review
      5.5.1. Search for sessions
         5.5.1.1. By date
         5.5.1.2. By session name
         5.5.1.3. By test type
      5.5.2. Open session
         5.5.2.1. Displays session data (as listed in 5.4.3)
      5.5.3. Save as .csv file
      5.5.4. Print session results (.pdf)
   5.6. Delete session (remove a session from the database)
   5.7. Settings—PC Application will allow the input and editing of the following control values:
      5.7.1. Initial Continuous Force Time—allowable range 0.1 to x.x seconds
      5.7.2. Initial Continuous Force Value—xxx grams
      5.7.3. Threshold Met Force Time—0.1 to x.x seconds
      5.7.4. Threshold Met Force—xxxx grams
      5.7.5. Lock Maintained Time Delay—0.1 to x.x seconds
      5.7.6. Lock Maintained Force—xxxx grams
      5.7.7. Lock Maintained LED On Time—x seconds
      5.7.8. Lock Lost LED On Time—x seconds

### Apparatus Definition—Parasympathetic Stimulation Mat

A mat provides parasympathetic stimulation and audible transmission of the agent sounds. The mat may be used instead of the aforementioned cuff. During testing, patient will lie on the mat. The test process will be the same as described above with the addition of the stimulation functionality listed below.
1. Mat is 175 cm (68.89 in) long and 62 cm (24.40 in) wide.
2. Mat is 1″ (2.54 cm) foam padding and cotton covering for patient comfort (see FIG. 1). Additional pillows or towels may be used to provide patient maximum comfort and head support.
3. Mat is connected to the control computer via USB connection.
4. Mat has an external power supply to power the vibration motors.
5. Mat is controlled per selections made by the practitioner in the computer application.
6. Mat has a speaker embedded at the upper portion of the mat for audible agent signal transmission
   6.1. Speaker is embedded in the foam on the upper portion of the mat (near the head) on the side where control wiring enters the mat
7. Mat has two rows of massage/vibration motors embedded within.
8. Vibration motors are positioned in the mat as follows:
   8.1. Beginning 35 cm (13.77 in) from the top of the mat
   8.2. Spaced 3 cm (1.18 in) apart
   8.3. Extending a total of 60 cm (23.62 in) down the mat
   8.4. Final 20 cm (20.87 in) of vibration motors are grouped for separate control to enable flexible use for varying sizes of patients.
9. Practitioner is able to control the vibration motors via the computer application to target specific nerve bundles. Vibration motors are controllable in 4 separate groups.
10. Practitioner is able to control the intensity/strength of the vibration motors via the computer application.
11. Stimulation can be selected to run only for a selected time period during the signal transmission (3-5 seconds), or can be set to run continuously.

It again, should be noted that the stimulation can be administered manually or via the mat.

It should be understood that the present invention is a method of training a living body of a patient, such that the following steps would be performed: positioning a speaker in the proximity of the patient; transmitting, via a computer, representational signals from the speaker, each signal representing a corresponding irritant; and administering the representational signals to the patient. The present invention also calls for stimulating the sympathetic ganglia when administering the representational signals via the speaker. Also, according to the present invention, one would ensure that converting each signal into a digital format is carried out. Further, the present invention calls for storing and matching each signal with the corresponding offending agent in a computer database.

Additionally, according to the present invention, one would place the speaker onto a cuff, the cuff configured to secure to an extremity of the patient. Moreover, the present invention can be viewed as positioning a speaker in the proximity of the patient; playing a signal from the speaker toward the patient, the signal matched with a corresponding irritant; and stimulating sympathetic ganglia locations while playing the signal from the speaker. Furthermore, the present invention should be viewed as positioning a speaker in the proximity of the patient; be it in within a mat or a cuff, playing, via a computer, signals from the speaker in the proximity of the patient's ears, each signal matched with a corresponding substance; facing the speaker toward the body of the patient; converting each signal into a digital format via a computer; storing and matching each signal with the corresponding substance in a computer database; placing the speaker onto a cuff or matt, the cuff or matt configured to play the signal in the proximity of the patient's ears. It should be understood that motor activity is measured before treatment to show an action schema of “retreat”. Stimulation is administered to the sympathetic ganglia until the motor activity is restored to normal capacity, indicating the perception of the substance has been modified. It should be noted that stimulation may be administered manually, or via the mat.

I claim:

1. A method of training a living body of a patient, comprising:
positioning a speaker in the proximity of the patient;
playing, via a computer, representational signals from the speaker, each signal representing a corresponding irritant; and
administering representational signals to the patient.

2. The method of claim 1, further comprising stimulating the sympathetic ganglia when administering the representational signals towards the patient via the speaker.

3. The method of claim 1, further comprising converting the signal into a digital audio format.

4. The method of claim 1, further comprising storing and matching each signal with the corresponding offending agent in a computer database.

5. The method of claim 1, further comprising placing the speaker into a mat.

6. A method of training a living body of a patient, comprising:
positioning a speaker in the proximity of the patient;
playing a signal from the speaker toward the patient, the signal matched with a corresponding irritant; and
stimulating sympathetic ganglia locations while transmitting the signal from the speaker.

7. A method of training a living body of a patient, comprising:
positioning a speaker in the proximity of the patient;
playing a signal from the speaker in the proximity of the patient, each signal matched with a corresponding substance;
facing the speaker toward the body of the patient;
converting each signal into a digital audio format via a computer;
storing and matching each signal with the corresponding substance in a computer database; and
placing the speaker into a mat, the mat configured to be laid on by the patient.
